(19) United States
(12) Patent Application Publication
(10) Pub. No.: US 2019/0031633 A1
(43) Pub. Date: Jan. 31, 2019

(54) LOW PRESSURE, HIGH TEMPERATURE PROCESS FOR FORMING 2,5-FURANDICARBOXYLIC ACID DIALKYL ESTER

(71) Applicant: E I DU PONT DE NEMOURS AND COMPANY, Wilmington, DE (US)

(72) Inventors: Judy H. Cipot-Wechsler, Kingston (CA); Pranit S. Metkar, Wilmington, DE (US); Ronnie Ozer, Arden, DE (US)

(21) Appl. No.: 15/742,946

(22) PCT Filed: Jul. 21, 2016
(86) PCT No.: PCT/US2016/043305

§ 371 Date: Jan. 9, 2018

Related U.S. Application Data
(60) Provisional application No. 62/196,803, filed on Jul. 24, 2015.

(51) Int. Cl. C07D 307/68

(52) U.S. Cl. CPC C07D 307/68

(57) ABSTRACT

Processes for preparing a dialkyl ester of 2,5-furan dicarboxylic acid are disclosed. The processes comprise contacting 2,5-furan dicarboxylic acid with excess alcohol and, optionally, a catalyst at a pressure in the range of between 1 bar and 21 bar and a temperature in the range of from 65° C. to 325° C. to form a vapor reaction product comprising an ester of 2,5-furan dicarboxylic acid, the alcohol and water; and distilling the vapor reaction product at a pressure in the range of from 0 bar to 3.5 bar at a temperature in the range of from 38° C. to 204° C. to separate the water and alcohol from the ester of 2,5-furan dicarboxylic acid.

### CROSS-REFERENCE TO RELATED APPLICATION

This application claims benefit of priority of U.S. Provisional Application No. 62/196,803 filed on Jul. 24, 2015, which is incorporated herein by reference in its entirety.

### FIELD OF THE DISCLOSURE

The present disclosure is directed towards an efficient process for the production of an ester of 2,5-furan dicarboxylic acid from the esterification of 2,5-furan dicarboxylic acid (FDCA).

### BACKGROUND OF THE DISCLOSURE

Derivatives of furan are known as potentially being useful in many industries, for example, pharmaceuticals, as fuel components, and as precursors for plastics. It has been disclosed that biomass materials can be used as a raw material to produce furan derivatives that might be useful as intermediates. For example, various sources of biomass can be hydrolyzed to produce pentose and hexose sugars which can then be further processed to form furfural and hydroxymethyl furfural (HMF).

In order to be industrially useful, the furfural and HMF must be efficiently processed to the desired materials. One useful product is furan dicarboxylic acid which can be further processed to form various polymers, including polyesters. Polyesters comprising the furan ring may have useful properties and could provide a replacement or partial replacement for polyesters derived from terephthalic acid. However, there is a continuing need for the efficient production of diesters of 2,5-furan dicarboxylic acid that can be processed into furan-containing polyesters.

### SUMMARY OF THE DISCLOSURE

In some embodiments, the process comprises:
a) contacting FDCA with excess alcohol and, optionally, a catalyst at a pressure in the range of between 1 bar and 21 bar and a temperature in the range of from 65° C. to 325° C. to form a vapor reaction product comprising an ester of FDCA, alcohol and water; and
b) distilling the vapor reaction product comprising the ester of FDCA at a pressure in the range of from 0 bar to 3.5 bar at a temperature in the range of from 38° C. to 204° C. to separate the water and alcohol from the ester of FDCA.

In other embodiments, the pressure of step a) is in the range of from 1 bar to 6 bar.

In some embodiments, the catalyst, if present, is cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst, or a combination thereof.

In other embodiments, the solid acid catalyst is a heterogeneous heteropolyacid, a salt of a heterogeneous heteropolyacid, sulfonic acid functionalized polymer, a cation exchange resin, a fluorinated sulfonic acid polymer, silica, titania, alumina, sulfated titania, sulfated zirconia, kaolinite, bentonite, attapulgite, montmorillonite, faujasite, beta zeolite, mordenite, or a combination thereof. In some embodiments, the solid acid catalyst comprises metal oxides, mixed metal oxides, metal sulfides, metal sulfates, metal sulfonates, metal nitrates, metal phosphates, metal phosphonates, metal molybdates, metal tungstates, metal borates, metal acetates or a combination thereof, wherein the metal is a Group 1 through Group 12 element of the Periodic Table.

In other embodiments, the process further comprises:
c) purifying the ester of FDCA from step b).

In other embodiments, step c) purifying the ester is by a distilling step, a crystallizing step, or a combination thereof.

In still further embodiments, the purified ester of FDCA comprises less than 10 ppm of the alkyl ester of 5-formylfuran-2-carboxylic acid, less than 10 ppm of the monoalkyl ester of 2,5-furan dicarboxylic acid, and/or less than 10 ppm FDCA, as determined by HPLC analysis.

### DETAILED DESCRIPTION OF THE DISCLOSURE

The disclosures of all cited patent and non-patent literature are incorporated herein by reference in their entirety.

As used herein, the term “embodiment” or “disclosure” is not meant to be limiting, but applies generally to any of the embodiments defined in the claims or described herein. These terms are used interchangeably herein.

Unless otherwise disclosed, the terms “a” and “an” as used herein are intended to encompass one or more (i.e., at least one) of a referenced feature.

The features and advantages of the present disclosure will be more readily understood, by those of ordinary skill in the art from reading the following detailed description. It is to be appreciated that certain features of the disclosure, which are, for clarity, described above and below in the context of separate embodiments, may also be provided in combination in a single element. Conversely, various features of the disclosure that are, for brevity, described in the context of a single embodiment, may also be provided separately or in any sub-combination. In addition, references to the singular may also include the plural (for example, “a” and “an” may refer to one or more) unless the context specifically states otherwise.

The use of numerical values in the various ranges specified in this application, unless expressly indicated otherwise, are stated as approximations as though the minimum and maximum values within the stated ranges were both proceeded by the word “about”. In this manner, slight variations above and below the stated ranges can be used to achieve substantially the same results as values within the ranges. Also, the disclosure of these ranges is intended as a continuous range including each and every value between the minimum and maximum values.

As used herein:

The term “solid acid catalyst” refers to any solid material containing Brönsted and/or Lewis acid sites, and which is substantially undissolved by the reaction medium under ambient conditions.

The phrase “ester of FDCA” means a diester of furan dicarboxylic acid. In some embodiments, the diester of furan dicarboxylic acid is the diester of 2,5-furandicarboxylic acid.

The phrase “alcohol source” means a molecule which, in the presence of water and optionally an acid forms an alcohol.

The acronym FDCA means 2,5-furan dicarboxylic acid.

The acronym FDME means the dimethyl ester of 2,5-furan dicarboxylic acid.

The acronym FDMME means the monomethyl ester of 2,5-furan dicarboxylic acid.

The acronym FFME means the methyl ester of 5-formylfuran-2-carboxylic acid.

The disclosure relates to efficient processes for producing FDME.

The process comprises:
a) contacting 2,5-furan dicarboxylic acid (FDCA) with excess alcohol and, optionally, a catalyst at a temperature in the range of from 65° C. to 325° C. and a pressure in the range of between 1 bar and 21 bar and to form a vapor reaction product comprising an ester of FDCA, the alcohol and water; and
b) distilling the vapor reaction product comprising the ester of FDCA at a temperature in the range of from 38° C. to 204° C. and a pressure in the range of from 0 bar to 3.5 bar to separate the water and alcohol from the ester of FDCA.

The process comprises a first step, of contacting FDCA with excess alcohol and, optionally a catalyst. Step a) of the process can be carried out in any suitable vessel, for example, a batch reactor, a continuously stirred tank reactor or a plug flow reactor that can be maintained at a pressure in the range of from 1 bar to 21 bar and at a temperature in the range of from 65° C. to 325° C. The pressure and temperature are chosen such that the reactor comprises a liquid component and at least a portion of the contents of the reactor in the gas phase. In some embodiments, the pressure can be in the range of from 1 bar to 20 bar. In other embodiments, the pressure can be in the range of from 1 to 19 bar, or from 1 to 18 bar, or from 1 to 17 bar, or from 1 to 16 bar, or from 1 to 15 bar, or from 1 to 14 bar, or from 1 to 13 bar, or from 1 to 12 bar, or from 1 to 11 bar, or from 1 to 10 bar, or from 1 to 9 bar, or from 1 to 8 bar, or from 1 to 7 bar, or from 1 to 6 bar. The temperature can be in the range of from 65° C. to 325° C. As used herein, the temperature refers to the temperature of the liquid phase in the reactor. In some embodiments, the temperature can be in the range of from 65° C. to 250° C., or from 70° C. to 230° C., or from 75° C. to 220° C., or from 90° C. to 200° C., or from 100° C. to 200° C. In other embodiments, the temperature can be in the range of from 175° C. to 325° C., or from 200° C. to 325° C., or from 225° C. to 325° C., or from 250° C. to 325° C., or from 260° C. to 320° C., or from 270° C. to 310° C., or from 280° C. to 310° C., or from 290° C. to 310° C.

The alcohol can be an alcohol having in the range of from 1 to 12 carbon atoms, especially alkyl alcohols. Suitable alcohols can include, for example methanol, ethanol, propanol, butanol, pentanol, hexanol, heptanol, octanol, nonanol, decanol, undecanol, dodecanol or isomers thereof. In some embodiments, the alcohol has in the range of from 1 to 6 carbon atoms or in the range of from 1 to 4 carbon atoms or in the range of from 1 to 2 carbon atoms. In some embodiments, the alcohol is methanol and the ester of FDCA is FDME.

In some embodiments, the percentage of FDCA and alcohol that can be fed to the reactor can be expressed as a weight percentage of the FDCA based on the total amount of FDCA and the alcohol. For example, the weight of FDCA can be in the range of from 1 to 70 percent by weight, based on the total weight of the FDCA and the alcohol. Correspondingly, the alcohol can be present at a weight percentage of about 30 to 99 percent by weight, based on the total amount of FDCA and the alcohol. In other embodiments, the FDCA can be present in the range of from 2 to 60 percent, or from 5 to 50 percent, or from 10 to 50 percent, or from 15 to 50 percent, or from 20 to 50 percent by weight, wherein all percentages by weight are based on the total amount of FDCA and the alcohol.

In other embodiments, the ratio of alcohol to the FDCA can be expressed in a molar ratio wherein the molar ratio of the alcohol to the FDCA can be in the range of from 2.01:1 to 40:1. In other embodiments, the molar ratio of the alcohol to FDCA can be in the range of from 2.2:1 to 40:1, or 2.5:1 to 40:1, or 3:1 to 40:1, or 4:1 to 40:1, or 8:1 to 40:1, or 10:1 to 40:1, or 15:1 to 40:1, or 20:1 to 40:1, or 25:1 to 40:1, or 30:1 to 40:1.

At least a portion of the alcohol can be replaced with an alcohol source. The alcohol source is a molecule which, in the presence of water and optionally an acid forms an alcohol. In some embodiments, the alcohol source is an acetal, an orthoformate, an alkyl carbonate, a trialkyl borate, a cyclic ether comprising 3 or 4 atoms in the ring or a combination thereof. Suitable acetals can include, for example, dialkyl acetals, wherein the alkyl portion of the acetal comprises in the range of from 1 to 12 carbon atoms. In some embodiments, the acetal can be 1,1-dimethoxyethane (acetaldehyde dimethyl acetal), 2,2 dimethoxypropane (acetone dimethyl acetal), 1,1-diethoxyethane (acetaldehyde diethyl acetal), 2,2 diethoxypropane (acetone diethyl acetal). Suitable orthoformates can be, for example, trialkyl orthoformate wherein the alkyl group comprises in the range of from 1 to 12 carbon atoms. In some embodiments, the orthoester is trimethyl orthoformate or triethyl orthoformate. Suitable alkyl carbonates can be dialkyl carbonates wherein the alkyl portion comprises in the range of from 1 to 12 carbon atoms. In some embodiments, the dialkyl carbonate is dimethyl carbonate or diethyl carbonate. Suitable trialkyl borates can be, for example, trialkyl borates wherein the alkyl portion comprises in the range of from 1 to 12 carbon atoms. In some embodiments, the trialkyl borate is trimethyl borate or triethyl borate. A cyclic ether can also be used wherein the cyclic ether has 3 or 4 atoms in the ring. In some embodiments, the cyclic ether is ethylene oxide or oxetane.

The alcohol source can be used in the same molar ratios as the alcohol. In the above embodiments, an alcohol or an alcohol source can be used in the contacting step a). In further embodiments, combinations of the alcohol and the alcohol source can also be used. In some embodiments, the percentage by weight of the alcohol can be in the range of from 0.001 percent to 99.999 percent by weight, based on the total weight of the alcohol and the alcohol source. In other embodiments, the alcohol can be present at a percentage by weight in the range of from 1 to 99 percent, or from 5 to 95 percent, or from 10 to 90 percent, or from 20 to 80 percent, or from 30 to 70 percent, or from 40 to 60 percent, wherein the percentages by weight are based on the total weight of the alcohol and the alcohol source.

The contacting step a) can optionally be performed in the presence of a catalyst. If present, the catalyst can be cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst or a combination thereof. The metal acetates, chlorides and hydroxides can be used as the hydrated salts. In some embodiments, the catalyst can be cobalt (II) acetate, iron (II) chloride, iron (III) chloride, magnesium (II) acetate, magnesium hydroxide, zinc (II) acetate or a hydrate thereof. In still further embodiments, the catalyst can be iron (II) chloride, iron (III) chloride or a combination thereof. In other embodiments, the catalyst can be cobalt acetate. In another embodiment, the catalyst can be sulfuric acid. Combinations of any of the above catalysts may also be useful. If present, a catalyst can be used at a rate of 0.1 to 5.0 percent by weight, based on the total weight of the FDCA, alcohol and optionally the alcohol source, and the catalyst. In other embodiments, the amount of catalyst present can be in the range of from 0.2 to 4.0 or from 0.5 to 3.0 or from 0.75 to 2.0 or from 1.0 to 1.5 percent by weight, wherein the percentages by weight are based on the total amount of FDCA, methanol and the catalyst.

The catalyst can also be a solid acid catalyst having the thermal stability required to survive reaction conditions. The solid acid catalyst may be supported on at least one catalyst support. Examples of suitable solid acids include without limitation the following categories: 1) heterogeneous heteropolyacids (HPAs) and their salts, 2) natural or synthetic minerals (including both clays and zeolites), such as those containing alumina and/or silica, 3) cation exchange resins, 4) metal oxides, 5) mixed metal oxides, 6) metal salts such as metal sulfides, metal sulfates, metal sulfonates, metal nitrates, metal phosphates, metal phosphonates, metal molybdates, metal tungstates, metal borates or combinations thereof. The metal components of categories 4 to 6 may be selected from elements from Groups 1 through 12 of the Periodic Table of the Elements, as well as aluminum, chromium, tin, titanium, and zirconium. Examples include, without limitation, sulfated zirconia and sulfated titania.

Suitable HPAs include compounds of the general formula XₐM_{b}O_{c}^{q-}, where X is a heteroatom such as phosphorus, silicon, boron, aluminum, germanium, titanium, zirconium, cerium, cobalt or chromium, M is at least one transition metal such as tungsten, molybdenum, niobium, vanadium, or tantalum, and q, a, b, and c are individually selected whole numbers or fractions thereof. Nonlimiting examples of salts of HPAs include, for example, lithium, sodium, potassium, cesium, magnesium, barium, copper, gold and gallium, and ammonium salts. Examples of HPAs suitable for the disclosed process include, but are not limited to, tungstosilicic acid (H₄[SiW₁₂O₄₀].xH₂O), tungstophosphoric acid (H₃[PW₁₂O₄₀].xH₂O), molybdophosphoric acid (H₃[PMo₁₂O₄₀].xH₂O), molybdosilicic acid (H₄[SiMo₁₂O₄₀].xH₂O), vanadotungstosilicic acid (H₄₊ₙ[SiVₙW₁₂₋ₙO₄₀]*xH₂O), vanadotungstophosphoric acid (H₃₊ₙ[PVₙW₁₂₋ₙO₄₀].xH₂O), vanadomolybdophosphoric acid (H₃₊ₙ[PVₙMo₁₂₋ₙO₄₀].xH₂O), vanadomolybdosilicic acid (H₄+n[SiVₙMo₁₂₋ₙO₄₀].xH₂O), molybdotungstosilicic acid (H₄[SiMoₙW₁₂₋ₙO₄₀].xH₂O), molybdotungstophosphoric acid (H₃[PMoₙW₁₂₋ₙO₄₀]*xH₂O), wherein n in the formulas is an integer from 1 to 11 and x is an integer of 1 or more.

Natural clay minerals are well known in the art and include, without limitation, kaolinite, bentonite, attapulgite, and montmorillonite.

In an embodiment, the solid acid catalyst is a cation exchange resin that is a sulfonic acid functionalized polymer. Suitable cation exchange resins include, but are not limited to the following: styrene divinylbenzene copolymer-based strong cation exchange resins such as AMBERLYST™ and DOWEX® available from Dow Chemicals (Midland, Mich.) (for example, DOWEX® Monosphere M-31, AMBERLYST™ 15, AMBERLITE™ 120); CG resins available from Resintech, Inc. (West Berlin, N.J.); Lewatit resins such as MONOPLUS™ S 100H available from Sybron Chemicals Inc. (Birmingham, N.J.); fluorinated sulfonic acid polymers (these acids are partially or totally fluorinated hydrocarbon polymers containing pendant sulfonic acid groups, which may be partially or totally converted to the salt form) such as NAFION® perfluorinated sulfonic acid polymer, NAFION® Super Acid Catalyst (a bead-form strongly acidic resin which is a copolymer of tetrafluoroethylene and perfluoro-3,6-dioxa-4-methyl-7-octene sulfonyl fluoride, converted to either the proton (H⁺), or the metal salt form) available from DuPont Company (Wilmington, Del.).

In an embodiment, the solid acid catalyst is a supported acid catalyst. The support for the solid acid catalyst can be any solid substance that is inert under the reaction conditions including, but not limited to, oxides such as silica, alumina, titania, sulfated titania, and compounds thereof and combinations thereof; barium sulfate; calcium carbonate; zirconia; carbons, particularly acid washed carbon; and combinations thereof. Acid washed carbon is a carbon that has been washed with an acid, such as nitric acid, sulfuric acid or acetic acid, to remove impurities. The support can be in the form of powder, granules, pellets, or the like. The supported acid catalyst can be prepared by depositing the acid catalyst on the support by any number of methods well known to those skilled in the art of catalysis, such as spraying, soaking or physical mixing, followed by drying, calcination, and if necessary, activation through methods such as reduction or oxidation. The loading of the at least one acid catalyst on the at least one support is in the range of 0.1-20 weight percent based on the combined weights of the at least one acid catalyst and the at least one support. Certain acid catalysts perform better at low loadings such as 0.1-5%, whereas other acid catalysts are more likely to be useful at higher loadings such as 10-20%. In an embodiment, the acid catalyst is an unsupported catalyst having 100% acid catalyst with no support such as, pure zeolites and acidic ion exchange resins.

Examples of supported solid acid catalysts include, but are not limited to, phosphoric acid on silica, NAFION®, a sulfonated perfluorinated polymer, HPAs on silica, sulfated zirconia, and sulfated titania. In the case of NAFION® on silica, a loading of 12.5% is typical of commercial examples.

In another embodiment, the solid acid catalyst comprises a sulfonated divinylbenzene/styrene copolymer, such as AMBERLYST™ 70.

In one embodiment, the solid acid catalyst comprises a sulfonated perfluorinated polymer, such as NAFION® supported on silica (SiO₂).

In one embodiment, the solid acid catalyst comprises natural or synthetic minerals (including both clays and zeolites), such as those containing alumina and/or silica.

Zeolites suitable for use herein can be generally represented by the following formula M_{2/n}O.Al₂O₃.xSiO₂.yH₂O wherein M is a cation of valence n, x is greater than or equal to about 2, and y is a number determined by the porosity and the hydration state of the zeolite, generally from about 2 to about 8. In naturally occurring zeolites, M is principally represented by Na, Ca, K, Mg and Ba in proportions usually reflecting their approximate geochemical abundance. The cations M are loosely bound to the structure and can frequently be completely or partially replaced with other cations by conventional ion exchange.

The zeolite framework structure has corner-linked tetrahedra with Al or Si atoms at centers of the tetrahedra and oxygen atoms at the corners. Such tetrahedra are combined in a well-defined repeating structure comprising various combinations of 4-, 6-, 8-, 10-, and 12-membered rings. The resulting framework structure is a pore network of regular channels and cages that is useful for separation. Pore dimensions are determined by the geometry of the aluminosilicate tetrahedra forming the zeolite channels or cages, with nominal openings of about 0.26 nm for 6-member rings, about 0.40 nm for 8-member rings, about 0.55 nm for 10-member rings, and about 0.74 nm for 12-member rings (these numbers assume the ionic radii for oxygen). Zeolites with the largest pores, being 8-member rings, 10-member rings, and 12-member rings, are frequently considered small, medium and large pore zeolites, respectively.

In a zeolite, the term “silicon to aluminum ratio” or, equivalently, “Si/Al ratio” means the ratio of silicon atoms to aluminum atoms. Pore dimensions are critical to the performance of these materials in catalytic and separation applications, since this characteristic determines whether molecules of certain size can enter and exit the zeolite framework.

In practice, it has been observed that very slight decreases in ring dimensions can effectively hinder or block movement of particular molecular species through the zeolite structure. The effective pore dimensions that control access to the interior of the zeolites are determined not only by the geometric dimensions of the tetrahedra forming the pore opening, but also by the presence or absence of ions in or near the pore. For example, in the case of zeolite type A, access can be restricted by monovalent ions, such as Na⁺ or K⁺, which are situated in or near 8-member ring openings as well as 6-member ring openings. Access can be enhanced by divalent ions, such as Ca²⁺, which are situated only in or near 6-member ring openings. Thus, the potassium and sodium salts of zeolite A exhibit effective pore openings of about 0.3 nm and about 0.4 nm respectively, whereas the calcium salt of zeolite A has an effective pore opening of about 0.5 nm.

The presence or absence of ions in or near the pores, channels and/or cages can also significantly modify the accessible pore volume of the zeolite for sorbing materials. Representative examples of zeolites are (i) small pore zeolites such as NaA (LTA), CaA (LTA), Erionite (ERI), Rho (RHO), ZK-5 (KFI) and chabazite (CHA); (ii) medium pore zeolites such as ZSM-5 (MFI), ZSM-11 (MEL), ZSM-22 (TON), and ZSM-48 (*MRE); and (iii) large pore zeolites such as zeolite beta (BEA), faujasite (FAU), mordenite (MOR), zeolite L (LTL), NaX (FAU), NaY (FAU), DA-Y (FAU) and CaY (FAU). The letters in parentheses give the framework structure type of the zeolite.

Zeolites suitable for use herein include medium or large pore, acidic, hydrophobic zeolites, including without limitation ZSM-5, faujasites, beta, mordenite zeolites or mixtures thereof, having a high silicon to aluminum ratio, such as in the range of 5:1 to 400:1 or 5:1 to 200:1. Medium pore zeolites have a framework structure consisting of 10-membered rings with a pore size of about 0.5-0.6 nm. Large pore zeolites have a framework structure consisting of 12-membered rings with a pore size of about 0.65 to about 0.75 nm. Hydrophobic zeolites generally have Si/Al ratios greater than or equal to about 5, and the hydrophobicity generally increases with increasing Si/Al ratios. Other suitable zeolites include without limitation acidic large pore zeolites such as H—Y with Si/Al in the range of about 2.25 to 5.

The reactor can be any of reactor having one or more feed inlets and one or more vapor outlets, as well as the capability to stir or provide mixing for the contents of the reactor. Suitable reactors can include, for example, a tank reactor, a continuously stirred tank reactor, a plug flow reactor, a reactive distillation column or a Scheibel column.

Contacting FDCA with the alcohol under the conditions specified forms a vapor phase reaction product comprising the ester of FDCA, the alcohol and water. Additionally, the vapor phase reaction product can further include the monoalkyl ester of FDCA, the alkyl ester of 5-formylfuran-2-carboxylic acid, or a combination thereof. The vapor phase reaction product is distilled in a second step at a pressure in the range of from 0 bar to 3.5 bar at a temperature in the range of from 38° C. to 204° C. to separate the water and the alcohol from the ester of FDCA. In other embodiments, the pressure of the distillation can be in the range of from 0.5 bar to 3.0 bar, or from 1.0 bar to 2.5 bar. The alcohol and water that is removed from the vapor phase reaction product can be recycled using known methods and reused in the process. In some embodiments, this recycling step includes a step of removing water from the alcohol. If an alcohol source is used, then the vapor phase can also comprise one or more of the by-products from the hydrolysis of the alcohol source. For example, in the presence of water, trimethyl orthoformate is known to form methanol and methyl formate. Other hydrolysis products of the disclosed alcohol sources are well-known in the art and can be present in the vapor phase.

After the water and the alcohol, and optionally any by-products from the alcohol source, is removed from the vapor phase reaction product, the ester of FDCA can be used as is. However, the process can comprise a further step: c) purifying the ester of FDCA from step b). The purification step can be a distillation step, a crystallization step, or a combination thereof. In some embodiments, the purification step c) is a distillation step, wherein the distillation is performed at low pressure, for example, in the range of from less than 1 bar to 0.0001 bar. In other embodiments, the pressure can be in the range of from 0.75 bar to 0.001 bar, or from 0.5 bar to 0.01 bar. The purification step can also be a crystallization step wherein the ester of FDCA from step b) can be recrystallized from any of the known recrystallization solvents, for example, methanol, ethanol or propanol. In some embodiments, the purification step c) can be accomplished by the distillation step followed by the recrystallization step, or by the recrystallization step followed by the distillation step.

Any of the above distillation and/or recrystallization steps can result in an ester of FDCA containing less than 50 parts per million (ppm) of any one of the impurities, as determined by HPLC analysis. For example, the ester of FDCA from step c) can contain less than 50 ppm of the alkyl ester of 5-formylfuran-2-carboxylic acid, less than 50 ppm of the monoalkyl ester of 2,5-furan dicarboxylic acid and/or less than 50 ppm FDCA, as determined by HPLC analysis. In other embodiments, the ester of FDCA from step c) can contain less than 25 ppm of the alkyl ester of 5-formylfuran-2-carboxylic acid, less than 25 ppm of the monoalkyl ester of 2,5-furan dicarboxylic acid and/or less than 25 ppm FDCA. In still further embodiments, the ester of FDCA from step c) can contain less than 10 ppm of the alkyl ester of 5-formylfuran-2-carboxylic acid, less than 10 ppm the monoalkyl ester of 2,5-furan dicarboxylic acid and/or less than 10 ppm FDCA. Any of the monoalkyl ester of 2,5-furan dicarboxylic acid or FDCA remaining from the purification step c) can be recycled back in to step a).

Non-limiting examples of the process disclosed herein include:
1. A process comprising:
   a) contacting FDCA with excess alcohol and, optionally, a catalyst at a pressure in the range of between 1 bar and 21 bar and a temperature in the range of from 65° C. to 325° C. to form a vapor reaction product comprising an ester of FDCA, the alcohol and water; and
   b) distilling the vapor reaction product comprising the ester of FDCA at a pressure in the range of from 0 bar to 3.5 bar at a temperature in the range of from 38° C. to 204° C. to separate the water and alcohol from the ester of FDCA.
2. The process of embodiment 1 wherein the pressure of step a) is in the range of from 1 bar to 6 bar.
3. The process of embodiment 1 wherein the catalyst, if present, is cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (II) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst, or a combination thereof.
4. The process of embodiment 3, wherein the solid acid catalyst is a heterogeneous heteropolyacid, a salt of a heterogeneous heteropolyacid, sulfonic acid functionalized polymer, a cation exchange resin, a fluorinated sulfonic acid polymer, silica, titania, alumina, sulfated titania, sulfated zirconia, kaolinite, bentonite, attapulgite, montmorillonite, faujasite, beta zeolite, mordenite, or a combination thereof.
5. The process of embodiment 1 wherein the process further comprises:
   c) purifying the ester of FDCA from step b).
6. The process of embodiment 5, wherein step c) purifying the ester is by a distilling step, a crystallizing step, or a combination thereof.
7. The process of embodiment 1 wherein the purified ester of FDCA comprises less than 50 ppm of an alkyl ester of 5-formylfuran-2-carboxylic acid, less than 50 ppm a monoalkyl ester of 2,5-furan dicarboxylic acid and/or less than 50 ppm FDCA, as determined by HPLC analysis.
8. The process of embodiment 1 wherein at least a portion of the alcohol is replaced with an alcohol source.
9. The process of embodiment 1 wherein the alcohol is methanol.
10. The process of embodiment 3, wherein the solid acid catalyst comprises metal oxides, mixed metal oxides, metal sulfides, metal sulfates, metal sulfonates, metal nitrates, metal phosphates, metal phosphonates, metal molybdates, metal tungstates, metal borates, or a combination thereof, wherein the metal is a Group 1 through Group 12 element of the Periodic Table.
11. The process of embodiment 1, wherein the ester of 2,5-furan dicarboxylic acid is dimethyl ester of 2,5-furan dicarboxylic acid.
12. The process of embodiment 8, wherein the alcohol source is an acetal, an orthoformate, an alkyl carbonate, a trialkyl borate, a cyclic ether comprising 3 or 4 atoms in the ring, or a combination thereof.

### EXAMPLES

Unless otherwise noted, all ingredients used herein are available from the Sigma-Aldrich Company, St. Louis, Mo.

### FDCA (Sarchem Labs, 99.0%)

### FDME (Sarchem Labs, 99.0%)

### Methanol (Fisher Scientific, 99.8%)

### N,N-dimethylformamide (DMF) (Sigma Aldrich)

### Acetonitrile (Fisher Scientific, A955-1)

### Isopropanol (Fisher Scientific, A4641 L1)

The following abbreviations are used in the examples: “° C.” means degrees Celsius; “wt %” means weight percent; “g” means gram; “min” means minute(s); “μL” means microliter; “ppm” means microgram per gram, “μm” means micrometer; “mL” means milliliter; “mm” means millimeter and “mL/min” means milliliter per minute; “v/v” means volume for volume; “FFCA” means 5-formyl-2-furancarboxlyic acid, “FDCA” means 2,5-furandicarboxylic acid, “FDME” means 2,5-furandicarboxylic acid dimethyl ester, “FDMME” means monomethyl ester of 2,5-furan dicarboxylic acid; “TFA” means trifluoroacetic acid, “HPLC” means high pressure liquid chromatography.

### Test/General Methods

### HPLC Analysis

HPLC analysis was used as one means to measure the FDCA, FDMME & FDME contents of the product mixture. An Agilent 1200 series HPLC equipped with a ZORBAX™ SB-Aq column (4.6 mm×250 mm, 5 μm) and photodiode array detector was used for the analysis of the reaction samples. The wavelength used to monitor the reaction was 280 nm. The HPLC separation of FDME, FDCA and FDMME was achieved using a gradient method with a 1.0 mL/min flow rate combining two mobile phases: Mobile Phase A: 0.5% v/v TFA in water and Mobile Phase B: acetonitrile. The column was held at 60° C. and 2 μL injections of samples were performed. Analyzed samples were diluted to <0.1 wt % for components of interest in a 50:50 (v/v) acetonitrile/isopropanol solvent. The solvent composition and flow rates used for the gradient method are given in Table 1 with linear changes occurring over the corresponding step whenever the composition changes.

TABLE 1
Gradient program for HPLC

| | Start | Volume % Mobile | Volume % Mobile |
|---|---|---|---|
| | time | Phase B, at | Phase B, at End of |
| Step | (min) | Beginning of Step | Step |
| 1 | 0.0 | 0 | 0 |
| 2 | 6.0 | 0 | 80 |
| 3 | 20.0 | 80 | 80 |
| 4 | 25.0 | 80 | 0 |
| 5 | 25.1 | 0 | 0 |
| 6 | 30.0 | 0 | 0 |

Retention times were obtained by injecting analytical standards of each component onto the HPLC. The amount of the analyte in weight percent was typically determined by injection of two or more injections from a given prepared solution and averaging the area measured for the component using the OpenLAB CDS C.01.05 software. The solution analyzed by HPLC was generated by dilution of a measured mass of the reaction sample with a quantified mass of 50:50 (v/v) acetonitrile/isopropanol solvent. Quantification was performed by comparing the areas determined in the OpenLAB software to a linear external calibration curve at five or more starting material concentrations. Typical R² values for the fit of such linear calibration curves were in excess of 0.9997.

While the presented HPLC method was used for this analysis, it should be understood that other HPLC methods could discriminate between products, starting materials, intermediates, impurities, and solvent and can be used for this analysis. It should also be understood that while HPLC was used as a method of analysis in this work, other techniques such as gas chromatography could also be optionally used for quantification when employing appropriate derivatization and calibration as necessary.

### LAB Color Measurements

A Hunterlab COLORQUEST™ Spectrocolorimeter (Reston, Va.) was used to measure the color in a sample of 2,5-furandicarboxylic acid. Color numbers are measured as APHA values (Platinum-Cobalt System) according to ASTM D-1209. The “b*” color of FDCA is calculated from the UV/VIS spectra and computed by the instrument. Color is commonly expressed in terms of Hunter numbers which correspond to the lightness or darkness (“L”) of a sample, the color value (“a*”) on a red-green scale, and the color value (“b*”) on a yellow-blue scale. The reaction samples were dissolved in DMF to produce a 6 wt. % solution and analyzed for purity and color.

### Size Exclusion Chromatography (SEC) Method for Humins Analysis

A screening assay was developed to estimate the weight concentration of a soluble humin byproduct using Size Exclusion Chromatography (SEC). An Alliance 2695 chromatograph (Waters Corporation, Milford, Mass.) was coupled to a 2498 dual-channel UV/Visible detector (Waters Corporation). UV absorbance was collected at wavelengths of 280 and 450 nm. The stationary phase consisting of a 4 column set (SHODEX™ KD-801, KD-802, and two KD-806M) was kept at a constant temperature of 50° C. Dimethylacetamide with 0.5% (w/v) lithium chloride was used as mobile phase at a flow rate of 1 mL/min. Samples were prepared by dissolving or diluting in the mobile phase, followed by agitation at room temperature for 4 hours, filtering through a 0.45 μm PTFE filter (Pall, Fort Washington, N.Y.), and injecting 100 μL into the instrument. A calibration curve was constructed using humin byproduct isolated from an acid-catalyzed fructose dehydration reaction. Resulting humin concentration in research samples was determined by integrating any eluting peak (450 nm absorbance) in the humin region of the chromatogram and comparing peak area to the calibration curve. A lower limit of detection was found to be approximately 50 ng humins.

### Example 1

### Preparation of FDME

For each experiment in Table 2, 0.1 grams of FDCA, 1.5 grams of FDME, and 500 microliters (μl) of methanol were added to a 4 milliliter (mL) reactor tube with air and then sealed with a SWAGELOCK® tubing plug (316 stainless steel, pressure rating of 228.5 bar (3300 psig)). The sealed reaction vessel was heated to the desired temperature for the time shown in Table 2. After the allotted time, the reaction vessel was removed from the heating source and then immersed in cold water to quench the reaction. Once the vessel reached room temperature, the pressure was released and the contents of the vessel were removed and transferred to an aluminum pan. The resulting solids were dried overnight in air, followed by drying in a vacuum oven at 80° C. The dried solids were then analyzed via HPLC using the conditions specified above.

TABLE 2

| | Temp | Duration | | | |
|---|---|---|---|---|---|
| Sample | (° C.) | (min) | FDCA % | FDMME % | FDME % |
| 1.1 | 200 | 15 | 5.96 | 2.39 | 91.65 |
| 1.2 | 200 | 30 | 4.42 | 2.34 | 93.24 |
| 1.3 | 200 | 60 | 2.60 | 7.17 | 90.22 |
| 1.4 | 230 | 15 | 2.36 | 5.19 | 92.45 |
| 1.5 | 230 | 30 | 0.29 | 5.39 | 94.32 |
| 1.6 | 230 | 60 | 0.37 | 7.19 | 92.44 |
| 1.7 | 270 | 15 | 0.22 | 5.79 | 93.99 |
| 1.8 | 270 | 30 | 0.18 | 7.17 | 92.65 |
| 1.9 | 270 | 60 | 0.32 | 11.41 | 88.27 |

The results indicate the reaction rate increases as a function of temperature, exhibited by the disappearance of FDCA and the production of FDMME. Table 2 shows that the conversion of FDCA was nearly complete at 15 minutes at 270° C., at 30 minutes at 230° C., and far from complete at 200° C. The HPLC analysis revealed only the three indicated products in the dried solids, suggesting reaction stability at these temperatures.

### Example 2

### Catalyst Screening Results at High Methanol Loading

The same procedure as used in Example 1 was completed by loading each tube reactor with 0.4 g FDCA, 2.005 mL methanol and either 0.028 grams catalyst or no catalyst as shown in Tables 3 and 4, and heated in a sand bath.

TABLE 3
Catalyst Screening Experiments (conditions: 80% methanol,
20% FDCA, 60 minutes reaction duration)

| | | | | FDME | |
|---|---|---|---|---|---|
| Sample | Catalyst | Temp (° C.) | FDCA % | % | FDMME % |
| 2.1 | None | 200 | 15.46 | 34.67 | 49.87 |
| 2.2 | Iron (III) | 200 | 2.48 | 76.58 | 20.94 |
| | Chloride | | | | |
| 2.3 | Iron (III) | 200 | 2.55 | 72.06 | 25.39 |
| | Chloride | | | | |
| 2.4 | Cobalt (II) | 200 | 2.26 | 77.13 | 20.61 |
| | Acetate | | | | |
| 2.5 | Cobalt (II) | 200 | 1.62 | 81.14 | 17.24 |
| | Acetate | | | | |
| 2.6 | Manganese | 200 | 9.12 | 57.99 | 32.89 |
| | (II) Acetate | | | | |
| 2.7 | Manganese | 200 | 9.64 | 55.15 | 35.21 |
| | (II) Acetate | | | | |
| 2.8 | None | 220 | 1.24 | 78.63 | 20.13 |

TABLE 4
Catalyst Screening Experiments (conditions: 80% methanol,
20% FDCA, 30 minutes reaction duration)

| | | Temp | | | |
|---|---|---|---|---|---|
| Sample | Catalyst | (° C.) | FDCA % | FDME % | FDMME % |
| 3.1 | None | 200 | 23.94 | 27.89 | 48.17 |
| 3.2 | Iron (III) | 200 | 3.32 | 64.93 | 31.75 |
| | Chloride | | | | |
| 3.3 | Iron (III) | 200 | 4.72 | 63.05 | 32.23 |
| | Chloride | | | | |
| 3.4 | Cobalt (II) | 200 | 12.84 | 46.37 | 40.79 |
| | Acetate | | | | |
| 3.5 | Cobalt (II) | 200 | 11.09 | 49.75 | 39.16 |
| | Acetate | | | | |
| 3.6 | Manganese | 200 | 17.50 | 41.97 | 40.53 |
| | (II) Acetate | | | | |
| 3.7 | None | 220 | 7.10 | 34.13 | 58.76 |

In Table 3, after 60 minutes of reaction time, all of the catalysts show an improved rate of esterification compared to the uncatalyzed control at the same temperature (samples 2.2 to 2.7 compared to 2.1). This was best indicated by lower FDCA concentrations, which correspond to greater extent of reaction. The best catalysts in Table 3 (samples 2.2, 2.3, 2.4 and 2.5) show FDCA conversion similar to sample 2.8 which was uncatalyzed at 20° C. higher temperature.

In Table 4, after 30 minutes of reaction time, all of the catalysts showed an improved rate compared to uncatalyzed at the same temperature (200° C., samples 3.2 to 3.6 compared to sample 3.7). The best catalyst result was in samples 3.2 and 3.3 (iron (III) chloride) which at 200° C. showed better FDCA conversion than the uncatalyzed sample at 220° C. (sample 3.7).

### Example 3

### Catalyst Screening Results at Low Methanol Loading

The same procedure as used in Example 1 was completed by loading each tube reactor with 0.1 g FDCA, 1.8 g FDME, 0.125 ml methanol and the amount of catalyst shown in Tables 5, 6, and 7 for 15, 30 and 60 minutes and a reaction time at 230° C. For the phosphoric acid and sulfuric acid reactions, a solution of acid in methanol was used to more precisely add the small quantities needed to give 0.47% phosphoric acid in the reactor, or 0.1% sulfuric acid in the reactor.

TABLE 5
Catalyst Screening Experiments (conditions: 5% methanol, 5%
FDCA, 90% FDME using Sand Bath heating at 230° C.), 15 minutes
reaction time

| | | | | | Catalyst |
|---|---|---|---|---|---|
| Sample | Catalyst | FDCA % | FDME % | FDMME % | (grams) |
| 4.1 | None | 2.23 | 94.69 | 3.08 | 0.0 |
| 4.2 | Cobalt (II) | 1.72 | 93.79 | 4.50 | 0.026 |
| | Acetate | | | | |
| 4.3 | Iron (II) | 0.74 | 90.47 | 8.79 | 0.021 |
| | Chloride | | | | |
| 4.4 | Iron (III) | 0.80 | 88.91 | 10.29 | 0.029 |
| | Chloride | | | | |
| 4.5 | Magnesium | 4.23 | 92.84 | 2.94 | 0.053 |
| | (II) Acetate | | | | |
| 4.6 | Magnesium | 2.86 | 93.27 | 3.87 | 0.014 |
| | Hydroxide | | | | |
| 4.7 | Zinc (II) | 1.53 | 93.16 | 5.31 | 0.020 |
| | Acetate | | | | |
| 4.8 | Phosphoric | 1.18 | 92.59 | 6.23 | 0.009 |
| | Acid | | | | |
| 4.9 | Sulfuric Acid | 0.67 | 86.28 | 13.05 | 0.002 |

TABLE 6
Catalyst Screening Experiments (conditions: 5% methanol, 5%
FDCA, 90% FDME using Sand Bath heating at 230° C.), 30 minutes
reaction time

| | | | FDME | | Catalyst |
|---|---|---|---|---|---|
| Sample | Catalyst | FDCA % | % | FDMME % | (grams) |
| 5.1 | None | 1.09 | 93.85 | 5.06 | 0.0 |
| 5.2 | Cobalt (II) | 0.89 | 92.66 | 6.45 | 0.026 |
| | Acetate | | | | |
| 5.3 | Iron (II) Chloride | 0.72 | 88.50 | 10.78 | 0.021 |
| 5.4 | Iron (III) Chloride | 0.62 | 91.34 | 8.04 | 0.029 |
| 5.5 | Magnesium (II) | 0.93 | 93.72 | 5.35 | 0.027 |
| | Acetate | | | | |
| 5.6 | Magnesium | 1.03 | 93.57 | 5.41 | 0.014 |
| | Hydroxide | | | | |
| 5.7 | Zinc (II) Acetate | 0.68 | 92.53 | 6.79 | 0.020 |
| 5.8 | Phosphoric Acid | 0.18 | 91.82 | 8.00 | 0.009 |
| 5.9 | Sulfuric Acid | 1.57 | 76.04 | 22.40 | 0.002 |

TABLE 7
Catalyst Screening Experiments (conditions: 5% methanol, 5%
FDCA, 90% FDME using Sand Bath heating at 230° C.), 60 minutes
reaction time

| | | | | | Catalyst |
|---|---|---|---|---|---|
| Sample | Catalyst | FDCA % | FDME % | FDMME % | (grams) |
| 6.1 | None | 0.83 | 95.40 | 3.77 | 0.0 |
| 6.2 | Cobalt (II) | 0.91 | 91.61 | 7.47 | 0.026 |
| | Acetate | | | | |
| 6.3 | Iron (II) | 0.22 | 91.67 | 8.11 | 0.021 |
| | Chloride | | | | |
| 6.4 | Iron (III) | 0.80 | 89.27 | 9.93 | 0.029 |
| | Chloride | | | | |
| 6.5 | Magnesium | 0.99 | 92.82 | 6.19 | 0.027 |
| | (II) Acetate | | | | |
| 6.6 | Magnesium | 1.07 | 98.50 | 0.43 | 0.014 |
| | Hydroxide | | | | |
| 6.7 | Zinc(II) | 0.13 | 93.13 | 6.74 | 0.020 |
| | Acetate | | | | |
| 6.8 | Phosphoric | 0.19 | 90.60 | 9.21 | 0.009 |
| | Acid | | | | |
| 6.9 | Sulfuric Acid | 2.40 | 70.83 | 26.78 | 0.002 |

Tables 5, 6, and 7 show the low methanol catalyst screening results at 15, 30, and 60 minutes. It is more difficult to see changes in the reaction results at these low levels of methanol in a small batch vessel. The catalytic effect is best shown in the production of the FDMME which can be made from transesterification of FDCA and FDME. The highest production of FDMME was shown with sulfuric acid and iron (III) chloride.

### Example 4

### Stability of FDME at High Temperature

The stability of FDME was studied in the presence of FDCA and methanol. This study was carried out in a 75 mL mini Parr reactor model 5050 equipped with an IKA RCT Basic hotplate stirrer. 24 g FDME, 0.5 g FDCA, 0.5 g methanol and a TFE stir bar was added to the reactor. The reactor was placed in an aluminum block and kept insulated. The reactor was then purged a minimum of 5 times with nitrogen. At room temperature, 300 psi of N₂ was introduced in the reactor head. The reactor was then heated to a desired temperature and both the temperature and pressure were monitored. After 1 hr, the reactor was allowed to cool to room temperature and the pressure was released. The reactor contents were removed and analyzed using different methods as stated below. These experiments were carried out at two different temperatures (270° C. & 300° C.) starting with a fresh sample of FDME (mixed with FDCA & methanol). The starting/original/control FDME sample is labelled as Sample A. The sample obtained at the end of 270° C. run is labelled as Sample 7.1. The sample obtained at the end of 300° C. run is labelled as Sample 7.2.

HPLC analysis and color analysis were used to measure the stability of FDME heated at high temperature. The solids collected at the end of above heating cycle were analyzed for FDME, FDMME, and FDCA. The results are summarized in Table 8.

TABLE 8
Results obtained after the analysis of solids collected at the
end of stability tests

| | Temp | | | Wt. % | Wt. % | Wt. % | Humins |
|---|---|---|---|---|---|---|---|
| Sample | (° C.) | L* | b* | FDME | FDMME | FDCA | (ppm) |
| A | — | 99.21 | 0.18 | 99.43 | 0.57 | 0 | <10 |
| 7.1 | 270 | 99.58 | 0.64 | 92.41 | 6.96 | 0.1 | <10 |
| 7.2 | 300 | 98.84 | 1.03 | 94.5 | 5.3 | 0.1 | <10 |

It is observed that the FDME is stable under the conditions tested above. The mass balance for furanics (FDME, FDMME, FDCA, etc.) was found to be very close to ˜100% which shows that there was no loss of furanics under the condition tested above. Also, the L* and b* values are very close to that of starting FDME material. The amount of humins formed during these experiment was below the detection limit of the SEC method (10 ppm). This indicates that the degradation of furanics to humins did not occur. Overall, FDME was found to be very stable under the conditions investigated.

What is claimed is:

1. A process comprising:
a) contacting 2,5-furan dicarboxylic acid with excess alcohol and, optionally, a catalyst at a pressure in the range of between 1 bar and 21 bar and a temperature in the range of from 65° C. to 325° C. to form a vapor reaction product comprising an ester of 2,5-furan dicarboxylic acid, the alcohol and water; and
b) distilling the vapor reaction product at a pressure in the range of from 0 bar to 3.5 bar at a temperature in the range of from 38° C. to 204° C. to separate the water and alcohol from the ester of 2,5-furan dicarboxylic acid.

2. The process of claim 1, wherein the pressure of step a) is in the range of from 1 bar to 6 bar.

3. The process of claim 1, wherein the catalyst, if present, is cobalt (II) acetate, iron (II) chloride, iron (III) chloride, iron (II) sulfate, iron (III) sulfate, iron (II) nitrate, iron (III) nitrate, iron (II) oxide, iron (III) oxide, iron (II) sulfide, iron (III) sulfide, iron (II) acetate, iron (III) acetate, magnesium (II) acetate, magnesium (II) hydroxide, manganese (II) acetate, phosphoric acid, sulfuric acid, zinc (II) acetate, zinc stearate, a solid acid catalyst, a zeolite solid catalyst, or a combination thereof.

4. The process of claim 3, wherein the solid acid catalyst is a heterogeneous heteropolyacid, a salt of a heterogeneous heteropolyacid, sulfonic acid functionalized polymer, a cation exchange resin, a fluorinated sulfonic acid polymer, silica, titania, alumina, sulfated titania, sulfated zirconia, kaolinite, bentonite, attapulgite, montmorillonite, faujasite, beta zeolite, mordenite, or a combination thereof.

5. The process of claim 1, wherein the process further comprises:
c) purifying the ester of 2,5-furan dicarboxylic acid from step b).

6. The process of claim 5, wherein c) purifying the ester is by a distilling step, a crystallizing step, or a combination thereof.

7. The process of claim 1, wherein the purified ester of 2,5-furan dicarboxylic acid comprises less than 50 ppm of an alkyl ester of 5-formylfuran-2-carboxylic acid, less than 50 ppm a monoalkyl ester of 2,5-furan dicarboxylic acid, and/or less than 50 ppm 2,5-furan dicarboxylic acid, as determined by HPLC analysis.

8. The process of claim 1 wherein at least a portion of the alcohol is replaced with an alcohol source.

9. The process of claim 1 wherein the alcohol is methanol.

10. The process of claim 8, wherein the alcohol source is an acetal, an orthoformate, an alkyl carbonate, a trialkyl borate, a cyclic ether comprising 3 or 4 atoms in the ring or a combination thereof.

11. The process of claim 1, wherein the ester of 2,5-furan dicarboxylic acid is dimethyl ester of 2,5-furan dicarboxylic acid.

12. The process of claim 3, wherein the solid acid catalyst comprises metal oxides, mixed metal oxides, metal sulfides, metal sulfates, metal sulfonates, metal nitrates, metal phosphates, metal phosphonates, metal molybdates, metal tungstates, metal borates, metal acetates or a combination thereof, wherein the metal is a Group 1 through Group 12 element of the Periodic Table.
